(19) United States
(12) Patent Application Publication
(10) Pub. No.: US 2017/0231562 A1
(43) Pub. Date: Aug. 17, 2017

(54) METHOD AND APPARATUS FOR ASSESSING DEGREE OF ALIGNMENT BETWEEN LIFE ACTIVITY RHYTHM AND CIRCADIAN RHYTHM

(71) Applicant: Samsung Electronics Co., Ltd., Suwon-si (KR)

(72) Inventors: Sangyun PARK, Hwaseong-si (KR); Younho KIM, Hwaseong-si (KR); Seungwoo NOH, Seongnam-si (KR)

(73) Assignee: Samsung Electronics Co., Ltd., Suwon-si (KR)

(21) Appl. No.: 15/350,187

(22) Filed: Nov. 14, 2016

(30) Foreign Application Priority Data
Feb. 16, 2016 (KR) 10-2016-0017557

(51) Int. Cl. A61B 5/00; A61B 5/026; A61B 5/11; A61B 5/01; A61B 5/024; A61B 5/145; A61B 5/021; A61B 8/06

(52) U.S. Cl. CPC A61B 5/4857; A61B 5/021; A61B 5/0059; A61B 5/0261; A61B 8/06; A61B 5/01; A61B 5/681; A61B 5/6804; A61B 5/024; A61B 5/0022; A61B 5/14546; A61B 5/4809; A61B 5/1118; A61B 5/7235

(57) ABSTRACT

Provided are a method and an apparatus for assessing a degree of alignment between circadian rhythm and life activity rhythm. The method involves sensing a biosignal of a user, deriving a circadian rhythm of the user based on a biosignal change pattern for the biosignal, and calculating a degree of alignment between the circadian rhythm and a life activity rhythm of the user.

### CROSS-REFERENCE TO RELATED APPLICATION(S)

This application claims the benefit under 35 USC 119(a) of Korean Patent Application No. 10-2016-0017557 filed on Feb. 16, 2016, in the Korean Intellectual Property Office, the entire disclosure of which is incorporated herein by reference for all purposes.

### BACKGROUND

1. Field

The following description relates to a method and an apparatus for providing a degree of alignment between a life activity rhythm and a circadian rhythm of a user.

2. Description of Related Art

In modern times, people are required to adjust their sleep cycles based on their busy schedule. Accordingly, physiological rhythms and sleep-wake cycles are often misaligned. Circadian rhythm refers to an internal physiological rhythm that encompasses biological processes that oscillate with respect to approximately 24 hours. The actual life activity rhythm, such as a sleep-wake cycle, of a person and the internal physiological rhythm, such as a circadian rhythm, of the person may be misaligned due to the busy schedule of the person. The misalignment between the physiological rhythm and the circadian rhythm may be especially great if the person is employed in a rotating shift work, is traveling abroad, is jet-lagged from an overseas trip, or has irregular sleeping habits do not match his or her external time cues. Continuing a lifestyle in which the misalignment persists between the life activity rhythm and the circadian rhythm may lead to various health issues.

### SUMMARY

This Summary is provided to introduce a selection of concepts in a simplified form that are further described below in the Detailed Description. This Summary is not intended to identify key features or essential features of the claimed subject matter, nor is it intended to be used as an aid in determining the scope of the claimed subject matter.

In one general aspect, a method of assessing a degree of alignment between circadian rhythm and life activity rhythm involves sensing a biosignal of a user, deriving a circadian rhythm of the user based on a biosignal change pattern for the biosignal, and calculating a degree of alignment between the circadian rhythm and a life activity rhythm of the user.

The general aspect of the method may further involve providing the degree of alignment to the user.

The sensing of the biosignal may involve sensing a blood pressure continuously for a predetermined duration.

The deriving of the circadian rhythm may involve determining whether the biosignal corresponds to an awake state or a sleep state, classifying the biosignal as a first biosignal corresponding to the awake state or a second biosignal corresponding to the sleep state, and analyzing the biosignal change pattern based on whether the biosignal is classified as the first biosignal or the second biosignal.

The determining of whether the biosignal corresponds to the awake state or the sleep state may involve determining a sleep-onset time at which the user begins to fall asleep, and determining, based on the sleep-onset time, whether the biosignal corresponds to the awake state or the sleep state.

The determining of the sleep-onset time may involve sensing whether the user moves using an acceleration sensor, and determining the sleep-onset time based on a result of the sensing.

The analyzing of the biosignal change pattern may involve analyzing at least one of a blood pressure drop pattern during sleep of the user or a blood pressure rise pattern during wakefulness of the user.

The general aspect of the method may further involve deriving the life activity rhythm using an acceleration sensor.

The general aspect of the method may further involve calculating an irregularity of the biosignal change pattern in the sleep state.

The calculating of the irregularity may involve aligning biosignal change patterns corresponding to a predetermined period of time based on the sleep-onset time, calculating a standard deviation for the aligned biosignal change patterns at each time, and calculating the irregularity based on the standard deviation.

The calculating of the degree of alignment may involve calculating the degree of alignment based on at least one of the irregularity or a ratio between the first biosignal and the second biosignal.

The providing of the degree of alignment may involve determining a sleep quality of the user based on the degree of alignment, and providing the determined sleep quality to the user.

In another general aspect, a non-transitory computer-readable medium stores instructions that, when executed by a processor, causes the processor to perform the general aspect of methods described above.

In yet another general aspect, an apparatus for assessing a degree of alignment between circadian rhythm and life activity rhythm includes a sensor configured to sense a biosignal of a user, and a processor configured to derive a circadian rhythm of the user based on a biosignal change pattern for the biosignal and calculate a degree of alignment between the circadian rhythm and a life activity rhythm of the user.

The processor may be further configured to provide the degree of alignment to the user.

The sensor may be configured to continually sense blood pressure as the biosignal.

The processor may be configured to determine whether the biosignal corresponds to an awake state or a sleep state of the user, to classify the biosignal as a first biosignal corresponding to the awake state or a second biosignal corresponding to the sleep state, and to analyze the biosignal change pattern based on whether the biosignal is the first biosignal or the second biosignal.

The processor may be configured to determine a sleep-onset time at which the user begins to fall asleep, and to determine, based on the sleep-onset time, whether the biosignal corresponds to the awake state or the sleep state.

The general aspect of the apparatus may further include an acceleration sensor configured to sense whether the user moves, and the processor may be configured to determine the sleep-onset time based on a result of the sensing of the acceleration sensor.

The processor may be configured to analyze at least one of a blood pressure drop pattern during sleep of the user or a blood pressure rise pattern during wakefulness of the user.

The general aspect of the apparatus may further include an acceleration sensor configured to sense whether the user moves, and the processor may be configured to derive the life activity rhythm based on a result of the sensing of the acceleration sensor.

The processor may be configured to calculate an irregularity of the biosignal change pattern in the sleep state, and to calculate the degree of alignment based on at least one of the irregularity or a ratio between the first biosignal and the second biosignal.

In another general aspect, a method of assessing a degree of alignment between circadian rhythm and sleep-wake cycle involves obtaining a biosignal of a user detected by a sensor; deriving, by a processor, a circadian rhythm of the user based on the biosignal; and calculating a degree of alignment between the circadian rhythm and a sleep-wake cycle of the user.

The general aspect of the method may further involve obtaining information regarding a movement of the user from an inertial sensor, and determining whether the biosignal corresponds to an awake state or a sleep state based on the information.

The calculating of the degree of alignment may involve analyzing a blood pressure change pattern during an awake state or a sleep state of the user.

Other features and aspects will be apparent from the following detailed description, the drawings, and the claims.

### BRIEF DESCRIPTION OF THE DRAWINGS

FIG. 1A illustrates an example of an apparatus for assessing a degree of alignment between circadian rhythm and life activity rhythm.

FIG. 1B illustrates examples of electronic devices that includes such an apparatus for assessing a degree of alignment between circadian rhythm and life activity rhythm.

FIG. 2 is a flowchart illustrating an example of a method of assessing a degree of alignment between circadian rhythm and life activity rhythm.

FIG. 3 is a flowchart illustrating an operation of deriving a circadian rhythm in an example of a method of assessing a degree of alignment according to FIG. 2.

FIG. 4 is a flowchart illustrating an example of an operation of determining whether a biosignal corresponds to an awake state or a sleep state.

FIG. 5 is a flowchart illustrating another example of a method of assessing a degree of alignment between circadian rhythm and life activity rhythm.

FIG. 6 illustrates examples of blood pressure changes during sleep.

FIG. 7 is a flowchart illustrating another example of a method of assessing a degree of alignment between circadian rhythm and life activity rhythm.

FIG. 8 is a flowchart illustrating an operation of calculating an irregularity of a biosignal change pattern in an example of a method according to FIG. 7.

FIG. 9 is a flowchart illustrating an operation of providing a degree of alignment to a user in an example of a method according to FIG. 7.

FIG. 10 is a diagram illustrating an example of a display format in which a degree of alignment between a circadian rhythm and a life activity rhythm of a user is provided to a user.

Throughout the drawings and the detailed description, the same reference numerals refer to the same elements. The drawings may not be to scale, and the relative size, proportions, and depiction of elements in the drawings may be exaggerated for clarity, illustration, and convenience.

### DETAILED DESCRIPTION

The following detailed description is provided to assist the reader in gaining a comprehensive understanding of the methods, apparatuses, and/or systems described herein. However, various changes, modifications, and equivalents of the methods, apparatuses, and/or systems described herein will be apparent after an understanding of this disclosure. For example, the sequences of operations described herein are merely examples, and are not limited to those set forth herein, but may be changed as will be apparent after an understanding of this disclosure, with the exception of operations necessarily occurring in a certain order. Also, descriptions of features that are known in the art may be omitted for increased clarity and conciseness.

The features described herein may be embodied in different forms, and are not to be construed as being limited to the examples described herein. Rather, the examples described herein have been provided merely to illustrate some of the many possible ways of implementing the methods, apparatuses, and/or systems described herein that will be apparent after an understanding of this disclosure.

Structural or functional descriptions of examples in the present disclosure are merely intended for the purpose of describing examples and the examples may be implemented in various forms and should not be construed as being limited to those described in the present disclosure.

Although terms of “first” or “second” are used to explain various components, the components are not limited to the terms. These terms are used only to distinguish one component from another component. For example, a “first” component may be referred to as a “second” component, or similarly, the “second” component may be referred to as the “first” component within the scope of the right according to the concept of the present disclosure.

It should be noted that if it is described in the specification that one component is “connected,” “coupled,” or “joined” to another component, a third component may be “connected,” “coupled,” and “joined” between the first and second components, although the first component may be directly connected, coupled or joined to the second component. In addition, it should be noted that if it is described in the specification that one component is “directly connected” or “directly joined” to another component, a third component may not be present therebetween. Likewise, expressions, for example, “between” and “immediately between” and “adjacent to” and “immediately adjacent to” may also be construed as described in the foregoing.

As used herein, the singular forms are intended to include the plural forms as well, unless the context clearly indicates otherwise. It will be further understood that the terms “comprises” and/or “comprising,” when used in this specification, specify the presence of stated features, integers, steps, operations, elements, components or a combination thereof, but do not preclude the presence or addition of one or more other features, integers, steps, operations, elements, components, and/or groups thereof.

Unless otherwise defined herein, all terms used herein including technical or scientific terms have the same meanings as those generally understood by one of ordinary skill in the art. Terms defined in dictionaries generally used should be construed to have meanings matching contextual meanings in the related art and are not to be construed as an ideal or excessively formal meaning unless otherwise defined herein.

In the following description, examples may be used to provide a degree of alignment between a circadian rhythm and a life activity rhythm of a user in daily life. Examples may be implemented as various products, for example, personal computers (PC), laptop computers, tablet computers, smartphones, smart home appliances, or wearable devices. For instance, examples may be employed to calculate a degree of alignment between a circadian rhythm and a life activity rhythm of a user based on a biosignal change pattern for a biosignal of the user in a smartphone, a mobile device, a smart home system or a wearable device. Also, examples may be applicable to a health care service based on a calculated blood pressure of a user. Hereinafter, examples will be described in detail below with reference to the accompanying drawings, and like reference numerals refer to the like elements throughout.

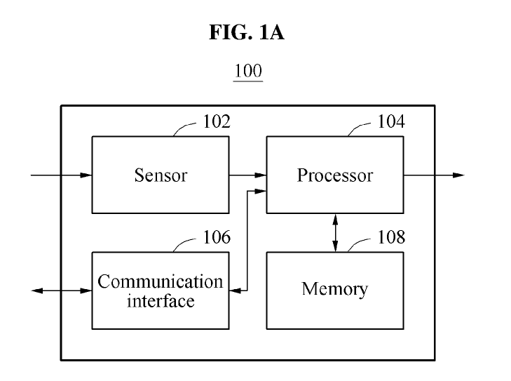

FIGS. 1A and 1B illustrate an example of an apparatus 100 (hereinafter, referred to as an “assessment apparatus” 100) for providing a degree of alignment, and examples of devices including the assessment apparatus 100.

Referring to FIG. 1A, the assessment apparatus 100 includes a sensor 102, a processor 104, a communication interface 106 and a memory 108. In this example, the sensor 102, the processor 104, the communication interface 106 and the memory 108 are implemented with hardware components. In this example, the sensor 102, the processor 104, the communication interface 106 and the memory 108 communicate with each other via a bus (not shown).

The sensor 102 senses a biosignal of a user. The sensor 102 may include a single sensor or a plurality of sensors. The sensor 102 may include, for example, a blood pressure sensor, a sensor configured to measure a photoplethysmography (PPG), a sensor configured to measure a change in a blood flow rate using an ultrasonic doppler and a laser doppler, or a body temperature sensor. The sensor 102 may include, for example, an inertial sensor or a global positioning system (GPS) sensor. An inertial sensor is configured to sense a movement of a user. Examples of inertial sensors include an acceleration sensor, a gyro sensor, a shock sensor, a tilt sensor and the like. The types of sensors that may be used, however, is not limited thereto, and other types of sensors may be used to implement the sensor 102.

Examples of biosignals that may be used by the assessment apparatus 100 include blood pressure, heartbeat, body temperature and/or the like. One or more biosignals may be continuously sensed over a predetermined time duration.

The processor 104 derives a circadian rhythm of the user based on a biosignal change pattern obtained from the biosignal sensed by the sensor 102. Circadian rhythm refers to a periodic phenomenon biochemically and physiologically occurring in a living organism. Circadian rhythm is involved in a pattern of biological activities such as sleeping, feeding, a brain-wave activity, hormone generation, or cell regeneration, for example. The circadian rhythm is also referred to as “biorhythm” or “biological clock.”

The processor 104 calculates a degree of alignment between the circadian rhythm of a user and a life activity rhythm of the user and provides the degree of alignment to the user or to other electronic devices or components. The processor 104 derives the life activity rhythm based on a sensing result of the sensor 102.

The life activity rhythm may be a daily activity rhythm that corresponds to activities or lack thereof actually performed by the user, such as a sleep-wake cycle. While the circadian rhythm may regulate the timing of sleepiness and wakefulness of a person, the sleep-wake cycle of the person may be influenced by various additional factors such as the person's schedule.

In this example, the processor 104 determines which one of an awake state and a sleep state corresponds to the biosignal. The biosignal may be, for example, a continuously sensed blood pressure. The processor 104 classifies the biosignal as a first biosignal corresponding to the awake state or a second biosignal corresponding to the sleep state based on a determination result. The awake state is understood as a non-sleep state, that is, a state in which a user is awake. The sleep state is understood as a state in which a user is sleeping. The processor 104 analyzes the biosignal change pattern based on the first biosignal and the second biosignal.

The processor 104 determines a sleep-onset time based on the sensing result of the sensor 102. The processor 104 classifies the biosignal as the first biosignal or the second biosignal, based on the sleep-onset time.

The processor 104 analyzes at least one of a blood pressure drop pattern during sleep of the user or a blood pressure rise pattern during wakefulness of the user.

The processor 104 calculates an irregularity of the biosignal change pattern in the sleep state of the user, and calculates the degree of alignment based on at least one of the irregularity or a ratio between the first biosignal and the second biosignal.

The communication interface 106 receives information from an external device, or provides the degree of alignment calculated by the processor 104 to the external device.

The memory 108 stores the biosignal sensed by the sensor 102 and the circadian rhythm and the degree of alignment derived by the processor 104. The memory 108 includes, for example, a volatile memory and a nonvolatile memory.

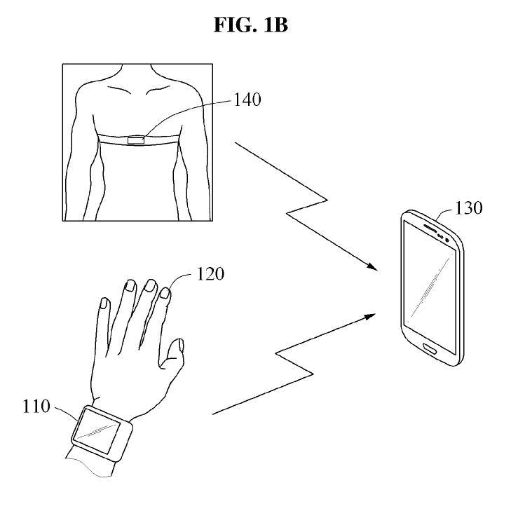

The processor 104 performs at least one method described with reference to FIGS. 2 through 10. The processor 104 executes a program and controls the assessment apparatus 100. A program code executed by the processor 104 is stored in the memory 108. The assessment apparatus 100 may be connected to an external device such as a PC or a network, for example, via an input/output device (not shown), and may exchange data with the external device.

At least one method described with reference to FIGS. 2 through 10 may be implemented as a chip or an application operating in a processor included in a tablet computer, a smartphone or a wearable device and may be included in a smartphone or a wearable device.

FIG. 1B illustrates wearable devices 110 and 140 and a mobile device 130 in which the assessment apparatus 100 may be mounted.

In an example, the assessment apparatus 100 is mounted in a wearable device 110 or a wearable device 140. In this example, the wearable device 110 is a wrist wearable device having a shape of a watch or a bracelet, and the wearable device 140 is a chest belt. However, in another example, the assessment apparatus 100 may be implemented in a wearable device having various other shapes.

In this example, when a user 120 is wearing the wearable devices 110 and 140 while performing daily activities, the assessment apparatus 100 determines a circadian rhythm of the user 120 based on a biosignal sensed from a wrist or a chest of the user 120. Also, the assessment apparatus 100 derives a life activity rhythm of the user 120 using a sensor that is included in one or more of the wearable devices 110 and 140. The sensor may be configured to sense a movement of the user 120. The assessment apparatus 100 calculates a degree of alignment between the circadian rhythm and the life activity rhythm of the user and provides the degree of alignment to the user. For example, the life activity rhythm may be a sleep-wake cycle of the user or other daily activity cycle, such as a meal-time cycle of the user.

In this example, the wearable devices 110 and 140 each include an assessment apparatus 100, and interoperate with the mobile device 130, thereby share data with the mobile device 130. For example, a blood pressure measured from the user 120, and the degree of alignment assessed by the assessment apparatus 100 may be transmitted to the mobile device 130 from the wearable devices 110 and 140.

In another example, the processor 104 of the assessment apparatus 100 is mounted in the mobile device 130, and the sensor 102 of the assessment apparatus 100 is mounted in each of the wearable devices 110 and 140. The wearable devices 110 and 140 are configured to be worn on a body part of the user 120, such as a wrist or a chest. The wearable devices 110 and 140 continuously measure a biosignal of the user 120 from the body part. The wearable devices 110 and 140 amplify and filter the measured biosignal. The wearable devices 110 and 140 transmit the measured biosignal to the mobile device 130. The assessment apparatus 100 included in the mobile device 130 provides a degree of alignment between the circadian rhythm and the life activity rhythm of the user 120 based on a heart rate received from each of the wearable devices 110 and 140.

The wearable devices 110 and 140 and the mobile device 130 are connected to each other via a wireless link. For example, the wearable devices 110 and 140 and the mobile device 130 each include a wireless Internet interface and a local area communication interface. The wireless Internet interface may include, for example, a wireless local area network (WLAN) interface, a wireless fidelity (Wi-Fi) direct interface, a Digital Living Network Alliance (DLNA) interface, a wireless broadband (WiBro) interface, a World Interoperability for Microwave Access (WiMAX) interface, or a high speed downlink packet access (HSDPA) interface. The local area communication interface may include, for example, a Bluetooth interface, a radio frequency identification (RFID) interface, an infrared data association (IrDA) interface, a ultra-wideband (UWB) interface, a ZigBee interface, or a near field communication (NFC) interface.

The mobile device 130 is implemented by, for example, a tablet computer, a smartphone, or a personal digital assistant (PDA). The mobile device 130 is, for example, a network device such as a server. Also, the mobile device 130 is, for example, a single server computer, a system similar to the server computer, at least one server bank, or a server “cloud” distributed between different geographical positions.

The mobile device 130 receives various biosignals in addition to the blood pressure, through the wearable devices 110 and 140 or other measurement devices.

FIG. 2 is a flowchart illustrating an example of a method of providing a degree of alignment. Referring to FIG. 2, in operation 210, an assessment apparatus senses a biosignal of a user. The sensed biosignal may be a biosignal that reflects a circadian rhythm. For example, the sensed biosignal may correspond to a concentration of hormone called melatonin in a body or in the blood, a core body temperature, a heartbeat, and/or a blood pressure. The user may not be conscious of the sensing of the biosignal of the user.

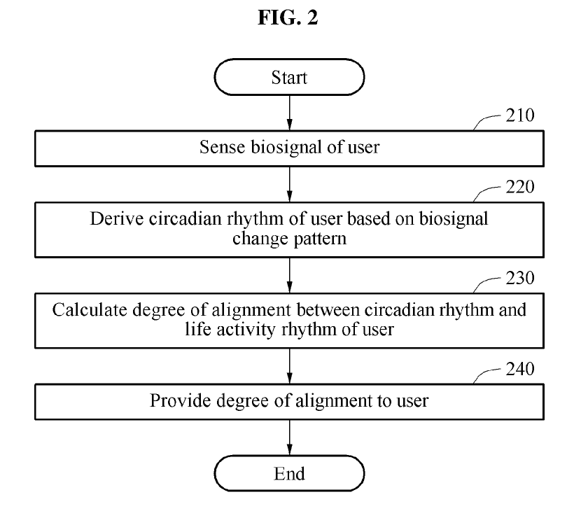

In operation 220, the assessment apparatus derives a circadian rhythm of the user based on a biosignal change pattern for the biosignal. Operation 220 is further described with reference to FIG. 3 below.

In operation 230, the assessment apparatus calculates a degree of alignment between the circadian rhythm derived in operation 220 and a life activity rhythm of the user. The life activity rhythm is derived using a GPS sensor or an inertial sensor, for example. Examples of an inertial sensor include an acceleration sensor, a gyro sensor, a shock sensor, a tilt sensor and the like. The inertial sensor may detect a movement of a user and provide information regarding the movement. The life activity rhythm may be, for example, an actual sleep-wake cycle of the user.

According to one example, the assessment apparatus calculates the degree of alignment in the form of, a synchronization index ranging from “1” to “10,” a graph, or a synchronization ratio (%).

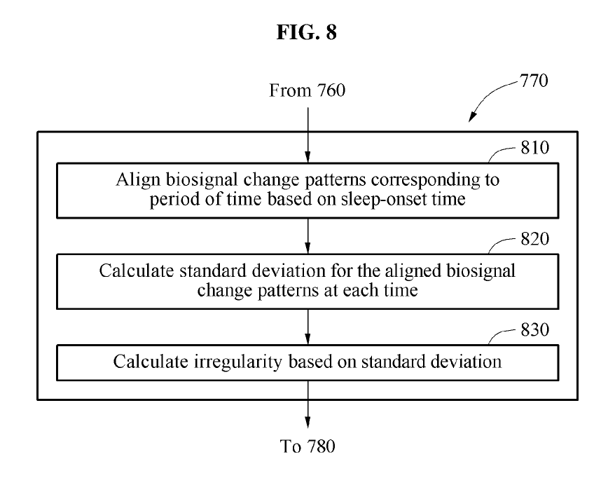

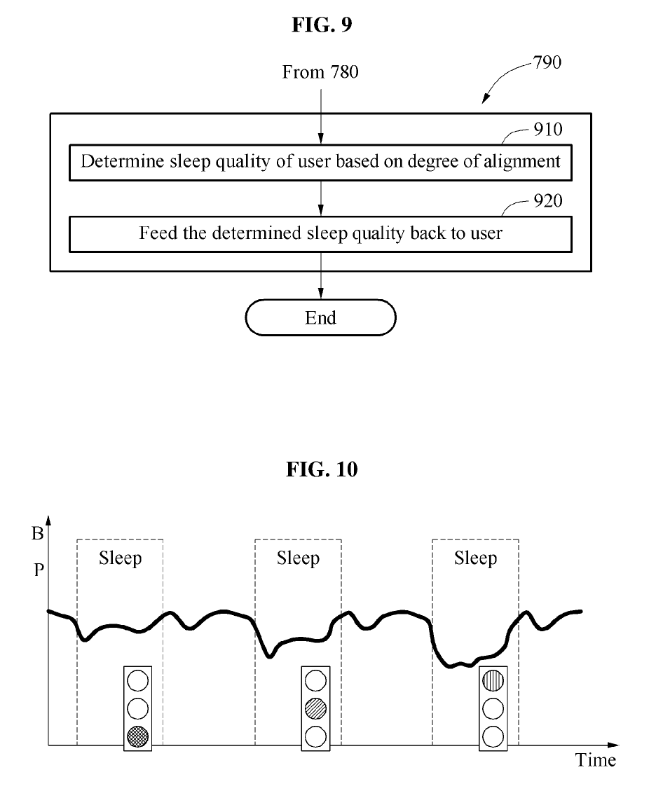

In operation 240, the assessment apparatus provides the degree of alignment to the user. An example in which the assessment apparatus provides the degree of alignment to the user will be further described with reference to FIGS. 9 and 10.

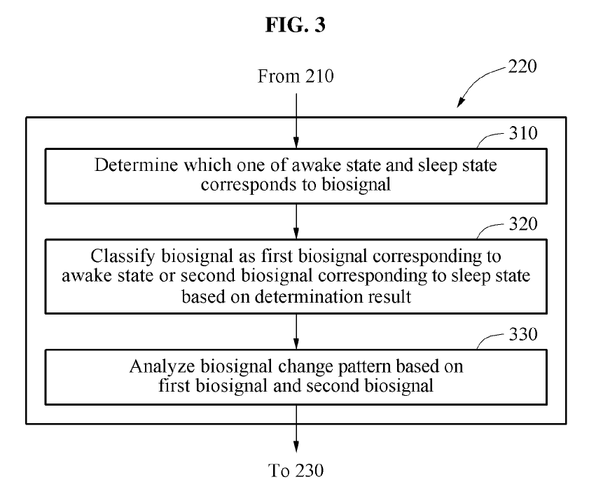

FIG. 3 is a flowchart illustrating operation 220 according to an example of a method illustrated in FIG. 2. Referring to FIG. 3, in operation 310, the assessment apparatus determines which one of an awake state and a sleep state of the user corresponds to the biosignal. An example of operation 310 to determine a state corresponding to a biosignal will be further described with reference to FIG. 4.

In operation 320, the assessment apparatus classifies the biosignal as a first biosignal corresponding to the awake state or a second biosignal corresponding to the sleep state based on a determination result of operation 310.

In operation 330, the assessment apparatus analyzes the biosignal change pattern based on the first biosignal and the second biosignal. To analyze the biosignal change pattern, the assessment apparatus may use, for example, a ratio between the first biosignal and the second biosignal. The assessment apparatus may analyze at least one of a blood pressure drop pattern during sleep of the user or a blood pressure rise pattern during wakefulness of the user. The assessment apparatus derives the circadian rhythm of the user based on the biosignal change pattern.

For example, a blood pressure of a user suddenly increases when the user wakes up in the morning and is maintained at a relatively high level during the day when the user engages in a lot of activities. The blood pressure gradually decreases in the evening, and noticeably decreases after an onset of sleep. When a blood pressure in a sleep state is not reduced by a predetermined level (for example, 5% through 10%) in comparison to a blood pressure in an awake state, a special high blood pressure called “non-dipper” is diagnosed with a poor prognosis related to cardiovascular diseases.

Research shows that a ratio between a blood pressure in a sleep state and a blood pressure in an awake state reflects a sleep quality among healthy people. Also, there is a prediction that a blood pressure in a sleep state after a shift work becomes higher than usual and is reduced to a normal level over time.

An irregular sleep cycle indicates an index representing a decrease in a sleep quality or a poor sleeping habit. Similarly, a great change in a biosignal change pattern during sleep every day may reflect a low sleep quality.

As described above, the assessment apparatus may calculate the degree of alignment between the circadian rhythm and the life activity rhythm of the user and a sleep quality of the user by determining whether the biosignal corresponds to the sleep state or the awake state and analyzing the biosignal change pattern based on a determination result.

For example, when a change pattern of a blood pressure in a sleep state of a user is observed during the time that the user is overcoming a jet lag resulting from traveling, it is found that significant daily changes in the pattern become almost similar. The assessment apparatus may also determine a degree of matching between a physiological rhythm and a physical sleep cycle of the user based on the pattern of the change in the blood pressure.

FIG. 4 is a flowchart illustrating an example of an operation of determining whether a biosignal corresponds to an awake state or a sleep state of a user. Referring to FIG. 4, in operation 410, an assessment apparatus senses whether a user is moving by using an acceleration sensor.

In operation 420, the assessment apparatus determines a sleep-onset time based on a sensing result of operation 410. The “sleep-onset time” is a point in time at which the user has initiated sleep and is understood as a point in time at which the user begins to fall asleep without a further movement. In an example, when a movement of the user sensed using the acceleration sensor in operation 410 is equal to or less than a preset first reference value, the assessment apparatus determines that the user is attempting to fall asleep. In another example, when the movement is equal to or less than a preset second reference value, the assessment apparatus determines that the user has begun to fall asleep. The first reference value is a value corresponding to a state in which the user slightly moves, for example, sitting, standing up or lying down. The second reference value is a value that is equal to or greater than a value (for example, a value of “0”) corresponding to a state in which the user does not move significantly, and is a value in a range less than that of the first reference value.

In operation 430, the assessment apparatus determines which one of an awake state and a sleep state corresponds to a biosignal based on the sleep-onset time determined in operation 420. For example, when the sleep-onset time is not determined in operation 420 because the user continues to move, the assessment apparatus determines the biosignal to correspond to the awake state.

When the sleep-onset time is determined in operation 420, the assessment apparatus determines the biosignal to correspond to the sleep state.

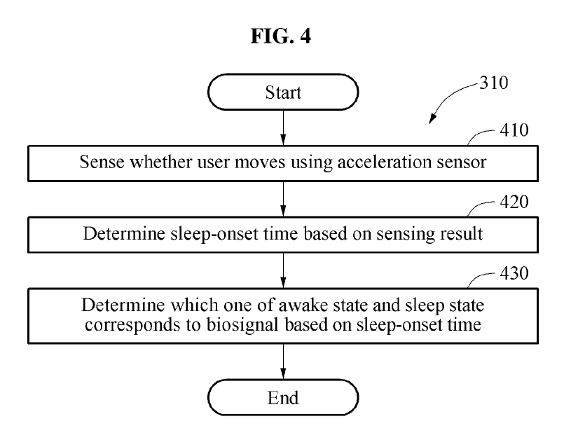

FIG. 5 is a flowchart illustrating another example of a method of providing a degree of alignment. Referring to FIG. 5, in operation 510, an assessment apparatus derives a life activity rhythm of a user using an acceleration sensor. To sense a movement of the user, the assessment apparatus may use, for example, a gyro sensor or a GPS sensor, in addition to the acceleration sensor. For example, the assessment apparatus may determine whether the user is sleeping, moving or eating or whether the user has a sedentary lifestyle, and may derive the life activity rhythm.

In operation 520, the assessment apparatus senses a biosignal of the user.

In operation 530, the assessment apparatus determines a sleep-onset time at which the user begins to fall asleep. For example, the assessment apparatus senses whether the user is moving by using the acceleration sensor, and determines the sleep-onset time based on a sensing result.

In operation 540, the assessment apparatus determines, based on the sleep-onset time, which one of an awake state and a sleep state corresponds to the biosignal.

In operation 550, the assessment apparatus classifies the biosignal as a first biosignal corresponding to the awake state or a second biosignal corresponding to the sleep state, based on a determination result of operation 540.

In operation 560, the assessment apparatus analyzes at least one of a blood pressure drop pattern during sleep of the user or a blood pressure rise pattern during wakefulness of the user based on the first biosignal and the second biosignal.

In operation 570, the assessment apparatus calculates a degree of alignment between a circadian rhythm and the life activity rhythm of the user based on the pattern analyzed in operation 560.

In operation 580, the assessment apparatus provides the degree of alignment to the user.

For example, the assessment apparatus may calculate a ratio between the first biosignal and the second biosignal, may categorize the degree of alignment as “good,” “satisfactory” or “poor” based on a range of the calculated ratio, and may provide the degree of alignment to the user.

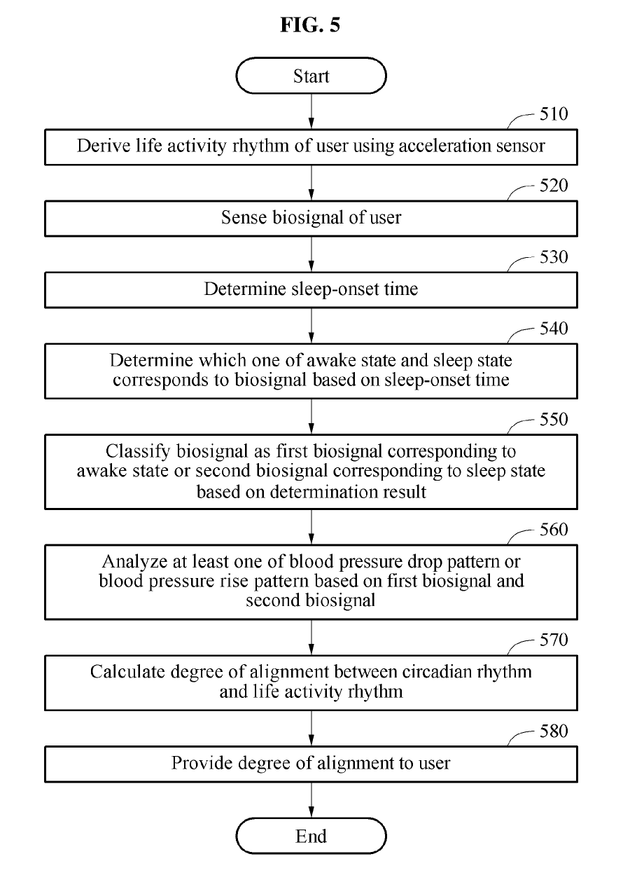

FIG. 6 illustrates examples of changes in blood pressure occurring during sleep. Referring to FIG. 6, graphs 610 and 630 represent a regular change in a blood pressure and an irregular change in the blood pressure while a single user is sleeping for a few days, respectively.

For example, when a user works in two or three different shifts during the night, has a temporary sleep problem due to a pregnancy or a jet lag from an overseas trip, or has a temporary sleep disorder due to a pregnancy, the blood pressure of the user temporarily and irregularly changes during sleep, as shown in the graph 630. When the user overcomes a jet lag or adapts to a shift work, the blood pressure of the graph 630 changes to the blood pressure of the graph 610.

Based on an irregularity of a blood pressure change pattern in the sleep state, a degree of alignment between a circadian rhythm and a life activity rhythm of the user may be calculated.

When a circadian rhythm is maintained after an onset of sleep even though a life activity rhythm of a user is disrupted, an irregularity of a blood pressure change pattern during sleep may not be serious. The assessment apparatus may quantify and provide a degree of alignment between a circadian rhythm and a life activity rhythm of the user or a disturbance degree of the circadian rhythm by combining the irregularity and a ratio between the first biosignal and the second biosignal.

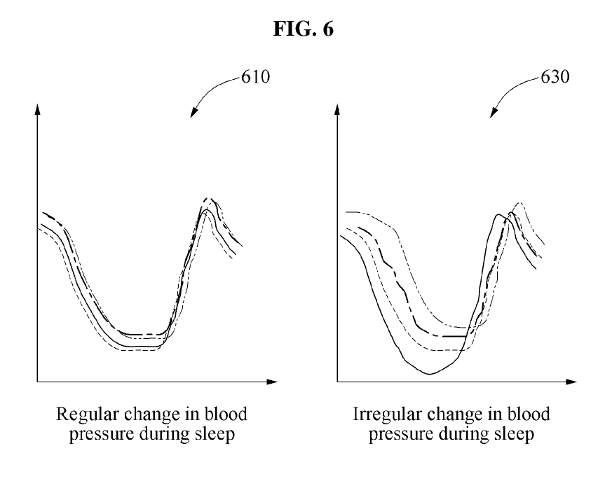

For example, the assessment apparatus may calculate a degree of alignment between a circadian rhythm and a life activity rhythm of a user based on a combination of two biosignals, for example, a heartbeat or a body temperature in a sleep state, as well as a blood pressure. An example of an operation of calculating the degree of alignment based on an irregularity of a biosignal change pattern is described with reference to FIG. 7.

FIG. 7 is a flowchart illustrating still another example of a method of assessing a degree of alignment between circadian rhythm and life activity rhythm. Operations 710 through 750 of FIG. 7 are the same as operations 510 through 550 of FIG. 5, and accordingly repetitive description thereof is omitted.

Referring to FIG. 7, in operation 760, an assessment apparatus analyzes a biosignal change pattern for a biosignal based on a first biosignal and a second biosignal.

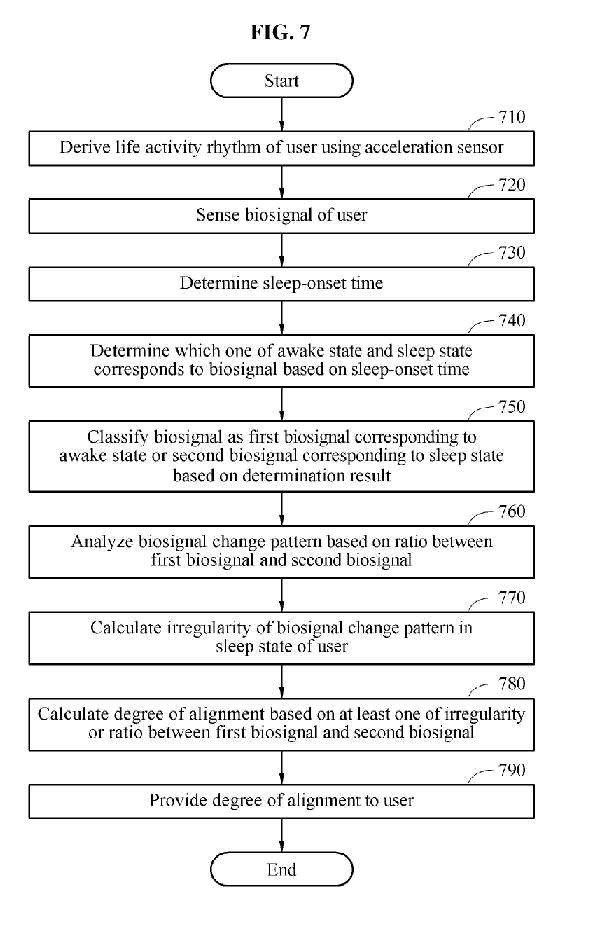

In operation 770, the assessment apparatus calculates an irregularity of the biosignal change pattern in a sleep state of a user. Operation 770 will be further described with reference to FIG. 8 below.

In operation 780, the assessment apparatus calculates a degree of alignment between a circadian rhythm and a life activity rhythm of the user based on at least one of a ratio between the first biosignal and the second biosignal or the irregularity calculated in operation 770. The assessment apparatus calculates the degree of alignment based on the irregularity calculated in operation 770, or based on a result of combining the irregularity and the ratio between the first biosignal and the second biosignal.

In operation 790, the assessment apparatus provides the degree of alignment calculated in operation 780. Operation 790 will be further described with reference to FIGS. 9 and 10 below.

FIG. 8 is a flowchart illustrating operation 770 according to an example of a method illustrated in FIG. 7. Referring to FIG. 8, in operation 810, the assessment apparatus aligns biosignal change patterns corresponding to a predetermined period of time based on a sleep-onset time.

In operation 820, the assessment apparatus calculates a standard deviation for the biosignal change patterns aligned in operation 810 at each time.

In operation 830, the assessment apparatus calculates the irregularity of the biosignal change pattern based on the standard deviation calculated in operation 820. For example, the assessment apparatus calculates the irregularity of the biosignal change pattern based on an average value that is based on the standard deviation calculated in operation 820.

FIG. 9 is a flowchart illustrating operation 790 according to an example of a method illustrated in FIG. 7. Referring to FIG. 9, in operation 910, the assessment apparatus determines a sleep quality of the user based on the degree of alignment between the circadian rhythm and the life activity rhythm. In an example, when the degree of alignment is greater than a predetermined criterion, the assessment apparatus determines that the sleep quality is high. In another example, when the degree of alignment is equal to or less than the criterion, the assessment apparatus determines that the sleep quality is low.

In operation 920, the assessment apparatus feeds the sleep quality determined in operation 910 back to the user. For example, the assessment apparatus may provide the user with the sleep quality information in a format illustrated in FIG. 10.

FIG. 10 is a diagram illustrating an example of a display format in which a degree of alignment between a circadian rhythm and a life activity rhythm of a user is provided to the user. Referring to FIG. 10, the degree of alignment is represented in a form of traffic lights.

According to this example, a user may select an icon for providing a degree of alignment between a circadian rhythm and a life activity rhythm, such as a sleep-wake cycle, of the user from a touch screen of a wearable device or a mobile device.

In response, an assessment apparatus displays the degree of alignment calculated using the above-described methods, on a display of the wearable device or the mobile device.

The assessment apparatus provides the degree of alignment by categorizing the degree of alignment as “good,” “satisfactory” or “poor.” The degree of alignment “good,” “satisfactory” or “poor” may be represented as green, orange or red traffic light. The green, orange and red traffic lights are a first circle, a second circle and a third circle of FIG. 10, respectively. Also, the assessment apparatus provides the user with the degree of alignment in a form of a percentage, a bar graph, or other expressible ratio forms.

As a non-exhaustive example only, a wearable device, a smartphone, a mobile device or a smart home system as described herein may be a terminal, such as a cellular phone, a wearable smart device (such as a ring, a watch, a pair of glasses, a bracelet, an ankle bracelet, a belt, a necklace, an earring, a headband, a helmet, or a device embedded in clothing), a portable personal computer (PC) (such as a laptop, a notebook, a subnotebook, a netbook, or an ultra-mobile PC (UMPC), a tablet PC (tablet), a phablet, a personal digital assistant (PDA), a digital camera, a portable game console, an MP3 player, a portable/personal multimedia player (PMP), a handheld e-book, a global positioning system (GPS) navigation device, or a sensor, or a stationary device, such as a desktop PC, a high-definition television (HDTV), a DVD player, a Blu-ray player, a set-top box, or a home appliance, or any other mobile or stationary device configured to perform wireless or network communication. In one example, a wearable device is a device that is designed to be mountable directly on the body of the user, such as a pair of glasses or a bracelet. In another example, a wearable device is any device that is mounted on the body of the user using an attaching device, such as a smart phone or a tablet attached to the arm of a user using an armband, or hung around the neck of the user using a lanyard.

The assessment apparatus, sensor, processor, communication interface, wearable device, mobile device and memory shown in FIGS. 1A and 1B that perform the operations described in this application are implemented by hardware components configured to perform the operations described in this application that are performed by the hardware components. Examples of hardware components that may be used to perform the operations described in this application where appropriate include controllers, sensors, generators, drivers, memories, comparators, arithmetic logic units, adders, subtractors, multipliers, dividers, integrators, antenna, display, non-transitory memory storage, and any other electronic components configured to perform the operations described in this application. In other examples, one or more of the hardware components that perform the operations described in this application are implemented by computing hardware, for example, by one or more processors or computers. A processor or computer may be implemented by one or more processing elements, such as an array of logic gates, a controller and an arithmetic logic unit, a digital signal processor, a microcomputer, a programmable logic controller, a field-programmable gate array, a programmable logic array, a microprocessor, or any other device or combination of devices that is configured to respond to and execute instructions in a defined manner to achieve a desired result. In one example, a processor or computer includes, or is connected to, one or more memories storing instructions or software that are executed by the processor or computer. Hardware components implemented by a processor or computer may execute instructions or software, such as an operating system (OS) and one or more software applications that run on the OS, to perform the operations described in this application. The hardware components may also access, manipulate, process, create, and store data in response to execution of the instructions or software. For simplicity, the singular term “processor” or “computer” may be used in the description of the examples described in this application, but in other examples multiple processors or computers may be used, or a processor or computer may include multiple processing elements, or multiple types of processing elements, or both. For example, a single hardware component or two or more hardware components may be implemented by a single processor, or two or more processors, or a processor and a controller. One or more hardware components may be implemented by one or more processors, or a processor and a controller, and one or more other hardware components may be implemented by one or more other processors, or another processor and another controller. One or more processors, or a processor and a controller, may implement a single hardware component, or two or more hardware components. A hardware component may have any one or more of different processing configurations, examples of which include a single processor, independent processors, parallel processors, single-instruction single-data (SISD) multiprocessing, single-instruction multiple-data (SIMD) multiprocessing, multiple-instruction single-data (MISD) multiprocessing, and multiple-instruction multiple-data (MIMD) multiprocessing.

The methods illustrated in FIGS. 2-5 and 7-9 that perform the operations described in this application are performed by computing hardware, for example, by one or more processors or computers, implemented as described above executing instructions or software to perform the operations described in this application that are performed by the methods. For example, a single operation or two or more operations may be performed by a single processor, or two or more processors, or a processor and a controller. One or more operations may be performed by one or more processors, or a processor and a controller, and one or more other operations may be performed by one or more other processors, or another processor and another controller. One or more processors, or a processor and a controller, may perform a single operation, or two or more operations. Further, processors distributed in two or more devices may perform a single processing operation. Herein, a processor may be refer to two or more processors distributed in two or more devices.

Instructions or software to control computing hardware, for example, one or more processors or computers, to implement the hardware components and perform the methods as described above may be written as computer programs, code segments, instructions or any combination thereof, for individually or collectively instructing or configuring the one or more processors or computers to operate as a machine or special-purpose computer to perform the operations that are performed by the hardware components and the methods as described above. In one example, the instructions or software include machine code that is directly executed by the one or more processors or computers, such as machine code produced by a compiler. In another example, the instructions or software includes higher-level code that is executed by the one or more processors or computer using an interpreter. The instructions or software may be written using any programming language based on the block diagrams and the flow charts illustrated in the drawings and the corresponding descriptions in the specification, which disclose algorithms for performing the operations that are performed by the hardware components and the methods as described above.

The instructions or software to control computing hardware, for example, one or more processors or computers, to implement the hardware components and perform the methods as described above, and any associated data, data files, and data structures, may be recorded, stored, or fixed in or on one or more non-transitory computer-readable storage media. Examples of a non-transitory computer-readable storage medium include read-only memory (ROM), random-access memory (RAM), flash memory, CD-ROMs, CD-Rs, CD+Rs, CD-RWs, CD+RWs, DVD-ROMs, DVD-Rs, DVD+Rs, DVD-RWs, DVD+RWs, DVD-RAMs, BD-ROMs, BD-Rs, BD-R LTHs, BD-REs, magnetic tapes, floppy disks, magneto-optical data storage devices, optical data storage devices, hard disks, solid-state disks, and any other device that is configured to store the instructions or software and any associated data, data files, and data structures in a non-transitory manner and provide the instructions or software and any associated data, data files, and data structures to one or more processors or computers so that the one or more processors or computers can execute the instructions. In one example, the instructions or software and any associated data, data files, and data structures are distributed over network-coupled computer systems so that the instructions and software and any associated data, data files, and data structures are stored, accessed, and executed in a distributed fashion by the one or more processors or computers.

While this disclosure includes specific examples, it will be apparent after an understanding of the disclosure of this application that various changes in form and details may be made in these examples without departing from the spirit and scope of the claims and their equivalents. The examples described herein are to be considered in a descriptive sense only, and not for purposes of limitation. Descriptions of features or aspects in each example are to be considered as being applicable to similar features or aspects in other examples. Suitable results may be achieved if the described techniques are performed in a different order, and/or if components in a described system, architecture, device, or circuit are combined in a different manner, and/or replaced or supplemented by other components or their equivalents. Therefore, the scope of the disclosure is defined not by the detailed description, but by the claims and their equivalents, and all variations within the scope of the claims and their equivalents are to be construed as being included in the disclosure.

What is claimed is:

1. A method of assessing a degree of alignment between circadian rhythm and life activity rhythm, the method comprising:
sensing a biosignal of a user;
deriving a circadian rhythm of the user based on a biosignal change pattern for the biosignal; and
calculating a degree of alignment between the circadian rhythm and a life activity rhythm of the user.

2. The method of claim 1, further comprising providing the degree of alignment to the user.

3. The method of claim 1, wherein the sensing of the biosignal comprises sensing a blood pressure continuously for a predetermined duration.

4. The method of claim 1, wherein the deriving of the circadian rhythm comprises:
determining whether the biosignal corresponds to an awake state or a sleep state;
classifying the biosignal as a first biosignal corresponding to the awake state or a second biosignal corresponding to the sleep state; and
analyzing the biosignal change pattern based on whether the biosignal is classified as the first biosignal or the second biosignal.

5. The method of claim 4, wherein the determining of whether the biosignal corresponds to the awake state or the sleep state comprises:
determining a sleep-onset time at which the user begins to fall asleep; and
determining, based on the sleep-onset time, whether the biosignal corresponds to the awake state or the sleep state.

6. The method of claim 5, wherein the determining of the sleep-onset time comprises sensing whether the user moves using an acceleration sensor; and
determining the sleep-onset time based on a result of the sensing.

7. The method of claim 4, wherein the analyzing of the biosignal change pattern comprises analyzing at least one of a blood pressure drop pattern during sleep of the user or a blood pressure rise pattern during wakefulness of the user.

8. The method of claim 1, further comprising:
deriving the life activity rhythm using an acceleration sensor.

9. The method of claim 5, further comprising:
calculating an irregularity of the biosignal change pattern in the sleep state.

10. The method of claim 9, wherein the calculating of the irregularity comprises:
aligning biosignal change patterns corresponding to a predetermined period of time based on the sleep-onset time;
calculating a standard deviation for the aligned biosignal change patterns at each time; and
calculating the irregularity based on the standard deviation.

11. The method of claim 9, wherein the calculating of the degree of alignment comprises calculating the degree of alignment based on at least one of the irregularity or a ratio between the first biosignal and the second biosignal.

12. The method of claim 2, wherein the providing of the degree of alignment comprises:
determining a sleep quality of the user based on the degree of alignment; and
providing the determined sleep quality to the user.

13. A non-transitory computer-readable medium storing instructions that, when executed by a processor, causes the processor to perform the method of claim 1.

14. An apparatus for assessing a degree of alignment between circadian rhythm and life activity rhythm, the apparatus comprising:
a sensor configured to sense a biosignal of a user; and
a processor configured to derive a circadian rhythm of the user based on a biosignal change pattern for the biosignal and calculate a degree of alignment between the circadian rhythm and a life activity rhythm of the user.

15. The apparatus of claim 14, wherein the processor is further configured to provide the degree of alignment to the user.

16. The apparatus of claim 14, wherein the sensor is configured to continually sense blood pressure as the biosignal.

17. The apparatus of claim 14, wherein the processor is configured to determine whether the biosignal corresponds to an awake state or a sleep state of the user, to classify the biosignal as a first biosignal corresponding to the awake state or a second biosignal corresponding to the sleep state, and to analyze the biosignal change pattern based on whether the biosignal is the first biosignal or the second biosignal.

18. The apparatus of claim 17, wherein the processor is configured to determine a sleep-onset time at which the user begins to fall asleep, and to determine, based on the sleep-onset time, whether the biosignal corresponds to the awake state or the sleep state.

19. The apparatus of claim 17, further comprising:
an acceleration sensor configured to sense whether the user moves,
wherein the processor is configured to determine the sleep-onset time based on a result of the sensing of the acceleration sensor.

20. The apparatus of claim 17, wherein the processor is configured to analyze at least one of a blood pressure drop pattern during sleep of the user or a blood pressure rise pattern during wakefulness of the user.

21. The apparatus of claim 14, further comprising:
an acceleration sensor configured to sense whether the user moves,
wherein the processor is configured to derive the life activity rhythm based on a result of the sensing of the acceleration sensor.

22. The apparatus of claim 17, wherein the processor is configured to calculate an irregularity of the biosignal change pattern in the sleep state, and to calculate the degree of alignment based on at least one of the irregularity or a ratio between the first biosignal and the second biosignal.

23. A method of assessing a degree of alignment between circadian rhythm and sleep-wake cycle, the method comprising:
obtaining a biosignal of a user detected by a sensor;
deriving, by a processor, a circadian rhythm of the user based on the biosignal; and
calculating a degree of alignment between the circadian rhythm and a sleep-wake cycle of the user.

24. The method of claim 23, further comprising:
obtaining information regarding a movement of the user from an inertial sensor; and
determining whether the biosignal corresponds to an awake state or a sleep state based on the information.

25. The method of claim 23, wherein the calculating of the degree of alignment comprises analyzing a blood pressure change pattern during an awake state or a sleep state of the user.
